(19) United States
(12) Patent Application Publication
(10) Pub. No.: US 2026/0083913 A1
(43) Pub. Date: Mar. 26, 2026

(54) Remote-Controlled Rotating Hand Mechanism in Aesthetic Medicine

(71) Applicant: Farshid Angootioshnari, London (GB)

(72) Inventor: Farshid Angootioshnari, London (GB)

(21) Appl. No.: 18/894,051

(22) Filed: Sep. 24, 2024

(51) Int. Cl. A61M 5/20

(52) U.S. Cl. CPC A61M 5/20; A61M 2205/18; A61M 2205/3375; A61M 2205/3553; A61M 2205/505; A61M 2205/581; A61M 2205/582; A61M 2205/583

(57) ABSTRACT

The present invention, RTA25 (Robotic Technology in Aesthetic), revolutionizes remote-controlled aesthetic treatments by integrating precision, safety, and operational efficiency. The RTA25 features a sophisticated rotating hand mechanism with multiple motors and gears, paired with an intuitive remote-control tool that mimics a syringe for precise maneuvering. Incorporating ultrasound technology, the system ensures real-time safety by monitoring critical zones during procedures. The comprehensive software platform enhances the experience with real-time video and audio interactions, robust client record management, and training modules for certifying practitioners. RTA25 breaks geographical barriers, setting new standards in aesthetic medicine by offering a transformative approach to modern aesthetic practices.

### FIELD OF THE INVENTION

The present invention relates to the field of aesthetic medicine, specifically to a novel robotic technology designed for remote-controlled aesthetic treatments, which includes mechanisms for precise injections and safety features.

### BACKGROUND OF THE INVENTION

In recent years, the field of aesthetic medicine has seen significant advancements in techniques and technologies designed to enhance the appearance and confidence of individuals. These advancements include minimally invasive procedures such as dermal fillers, Botox injections, and other cosmetic treatments. However, despite these innovations, the field continues to face several critical challenges that hinder its growth and accessibility.

The predominant concern in aesthetic medicine is the safety of procedures. Complications from injections, including nerve damage, vascular occlusions, and infections, pose significant risks. Traditional methods often rely heavily on the practitioner's skill and experience, leading to variations in safety and outcomes.

Highly skilled and reputable aesthetic practitioners are often concentrated in metropolitan areas. Patients living in remote or underserved regions face difficulties accessing quality aesthetic care. This geographical barrier limits the reach of practitioners and creates a disparity in the availability of services.

Frequent travel to different locations to meet client demand can lead to practitioner fatigue, reducing their overall efficiency and performance. This constant travel not only impacts the well-being of practitioners but also restricts the number of clients they can effectively serve.

Achieving consistent and precise results in aesthetic treatments is crucial. Manual injections may vary between sessions or practitioners, leading to inconsistent outcomes. The precision required in aesthetic procedures demands advanced tools to minimize human error.

Various tools and techniques have been developed to address some of these challenges. For example, automated injection devices aim to improve precision and consistency, while telemedicine platforms attempt to extend the reach of practitioners through virtual consultations.

While they can enhance precision, automated devices lack the adaptability and real-time responsiveness required in dynamic aesthetic treatments. Although useful for consultations, telemedicine cannot facilitate the actual administration of procedures, thereby still necessitating physical presence, which limits its effectiveness.

To overcome these challenges, we present RTA25 (Robotic Technology in Aesthetic), a state-of-the-art robotic system designed to revolutionize the practice of aesthetic medicine. RTA25 integrates advanced robotics, real-time remote control, and sophisticated software to offer a comprehensive solution that addresses safety, accessibility, and efficiency.

The primary innovation of RTA25 lies in its rotating hand mechanism, which allows for precise and versatile injections controlled remotely by an aesthetic doctor. This system not only ensures consistent and accurate administration of treatments but also significantly reduces the risks associated with manual injections.

Additionally, the inclusion of ultrasound technology enhances safety by providing real-time feedback and alerts to the practitioner, thus minimizing the risk of complications. The remote-control tool, designed to mimic a syringe, ensures that practitioners can perform procedures with the same level of control and familiarity as if they were physically present.

The RTA25 system also features a robust software platform that enables real-time interaction between the practitioner and the client through video and audio communication. This platform supports a central database for managing client information, treatment records, and before-and-after images, facilitating personalized and data-driven treatment plans.

RTA25 aims to democratize access to high-quality aesthetic treatments by eliminating geographical barriers and enabling practitioners to serve clients remotely. This innovation not only improves the convenience for practitioners and clients but also sets new standards for safety and precision in aesthetic medicine.

By addressing the key challenges faced by the industry, RTA25 represents a significant leap forward, offering a reliable, efficient, and safe solution for modern aesthetic practices. The integration of advanced robotics and real-time control technology positions RTA25 as a pioneering innovation in the ever-evolving landscape of aesthetic medicine.

### SUMMARY OF THE INVENTION

The present invention addresses significant challenges within the field of aesthetic medicine by introducing a novel robotic system, the RTA25, designed to enable remote-controlled aesthetic treatments with unparalleled precision and safety.

As the field of aesthetic medicine continues to expand, practitioners and clients are confronted with issues such as safety concerns, limited accessibility due to geographical constraints, and the physical and mental fatigue experienced by traveling practitioners. These challenges often result in inconsistent treatment outcomes and hinder the overall growth and reach of aesthetic services.

RTA25 is designed to overcome these obstacles by integrating advanced robotics technology with real-time remote-control capabilities. At the core of this system is the innovative rotating hand mechanism, equipped with an array of motors and gears that allow for precise, versatile injections in any required direction and at any depth. This mechanism is controlled remotely by practitioners using a specifically designed tool that resembles a traditional syringe, providing them with a high degree of control and familiarity. Communication between the remote-control tool and the robotic hand is facilitated through a secure wireless connection, ensuring real-time responsiveness and minimal latency.

To enhance safety, RTA25 incorporates ultrasound technology that continuously monitors the treatment area for potential danger zones, such as blood vessels or sensitive nerves. This real-time scanning capability provides immediate alerts to practitioners, helping to mitigate the risks of complications such as vascular occlusions or nerve damage. The addition of this ultrasound module underscores the commitment to patient safety and exemplifies the innovative approach of the RTA25 system.

Furthermore, the software integrated with RTA25 allows for seamless real-time interaction between the practitioner and the client. Through high-definition video and audio communication, practitioners can conduct consultations, monitor treatments, and adjust procedures in real-time as if they were physically present with the client. The software also features a central database for storing and managing comprehensive client information, including treatment histories and before-and-after images, which can be utilized to tailor personalized treatment plans and track progress over time.

### BRIEF DESCRIPTION OF DRAWINGS

FIG. 1 illustrates a surgical robotic arm designed for precision surgeries.

FIG. 2 depicts the three individual arms of the surgical robot.

FIG. 3 shows the movement and coordination functionality of the robotic system.

FIG. 4 displays the sensor holder used in the robotic arm.

FIG. 5 presents the harmonic drives that enable smooth and precise movements.

FIG. 6 demonstrates the servo motors providing controlled motion to the arms.

FIG. 7 shows the ultrasonic sensor holder for enhanced guidance.

FIG. 8 illustrates the arm designed for pinching, holding, and pressing tasks.

FIG. 9 depicts the arm specifically used for injection purposes.

### DETAILED DESCRIPTION OF THE INVENTION

The present invention, RTA25 (Robotic Technology in Aesthetic), is designed to address various challenges in aesthetic medicine by providing a sophisticated robotic system capable of performing remote-controlled aesthetic treatments with high precision and safety. The RTA25 system includes a reserved power supply and redundant network capabilities to ensure continuous operation and reliability. A backup battery system maintains full functionality during power outages, mitigating risks associated with sudden power failures. Dual-network capabilities prevent connectivity loss, ensuring seamless real-time control, video and audio interactions, and database management. These features enhance operational stability, safety, and reliability for aesthetic treatments. This detailed description provides an in-depth look into the components, functionalities, and operational processes of the RTA25 system.

The rotating hand mechanism is the core of the RTA25 system, designed to mimic the precise and versatile movements of a skilled aesthetic doctor's hand. The mechanism is equipped with multiple motors and gears that allow for rotations and movements in various planes, enabling the system to perform injections in any desired direction and at specific depths.

The rotating hand utilizes a combination of brushless motors and high-precision gears to achieve smooth and accurate movements. These components are controlled by advanced software algorithms that translate the practitioner's inputs from the remote-control tool into precise mechanical actions.

The rotating hand is compatible with various types of needles and syringes, which are securely mounted and can be easily replaced. The system accommodates different treatment requirements by adjusting the depth and angle of injections dynamically.

The remote-control tool is a critical component that enables practitioners to perform treatments remotely with a high degree of control and accuracy. It is designed to resemble a traditional syringe, providing a familiar interface that mimics the tactile feedback and responsiveness of manual injections.

The tool communicates with the rotating hand mechanism via a secure and reliable wireless connection, ensuring real-time responsiveness with minimal latency. The connection is encrypted to prevent any unauthorized access or interference. The tool includes controls for adjusting the depth and speed of injections, as well as directional movements. Practitioners can make real-time adjustments based on their visual and tactile feedback during the treatment process.

Safety in aesthetic treatments is paramount, and the RTA25 incorporates advanced ultrasound technology to enhance this aspect. The ultrasound module is integrated into the rotating hand mechanism and continuously scans the treatment area during procedures. The ultrasound module provides real-time imaging of the underlying tissues, identifying critical structures such as blood vessels and nerves that must be avoided during injections. The system generates immediate alerts if a danger zone is detected, allowing the practitioner to take corrective action promptly. This feature significantly reduces the risk of complications and enhances the overall safety of the procedure.

The RTA25 software platform is designed to facilitate seamless and interactive operation between the practitioner and the client. It includes several key functionalities that enhance the treatment process. The software supports high-definition video and audio communication, allowing practitioners to see and hear clients in real-time. This interaction is crucial for conducting thorough consultations, monitoring procedures, and making necessary adjustments during treatments.

The software includes a central database that stores comprehensive client information, including personal details, medical histories, treatment records, and before-and-after images. This database allows practitioners to tailor personalized treatment plans and track client progress over time.

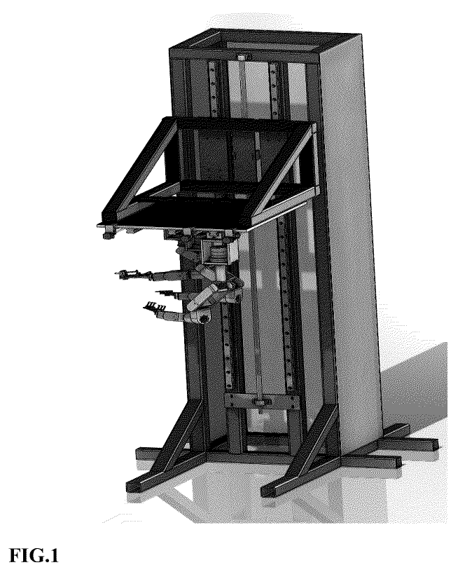

FIG. 1 is the surgical robotic arm illustrates the overarching design of the surgical robotic system. The main frame of this system is robust and designed to support all attached components securely. This frame is capable of vertical movement, allowing the entire assembly to be positioned at the correct height relative to the patient's treatment area. The main frame's mobility is enhanced by the inclusion of castors at its base, which allows for the system to be easily rolled and positioned as needed. Once the system is in the desired location, the castors can be locked to maintain stability during operations.

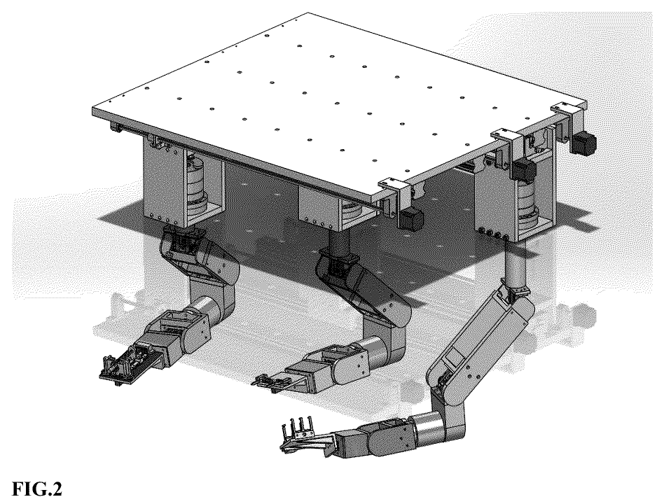

FIG. 2 is the three robotic arms three robotic arms are depicted, each with a 6-axis configuration. This multi-axis flexibility ensures that each arm can achieve a broad range of positions and angles necessary for various procedures. On the right, one arm is shown holding an ultrasonic sensor. This arm's main function is to scan and assess the condition of the skin, providing critical data to inform the treatment process. The middle arm, equipped with specialized pinching jaws, is responsible for holding and stabilizing the skin. This function is essential for ensuring that the skin remains still and taut during the injection process. The left arm, specifically designed for injections, holds and operates a syringe. This arm's precise movements are controlled to administer the requisite treatment accurately into the skin.

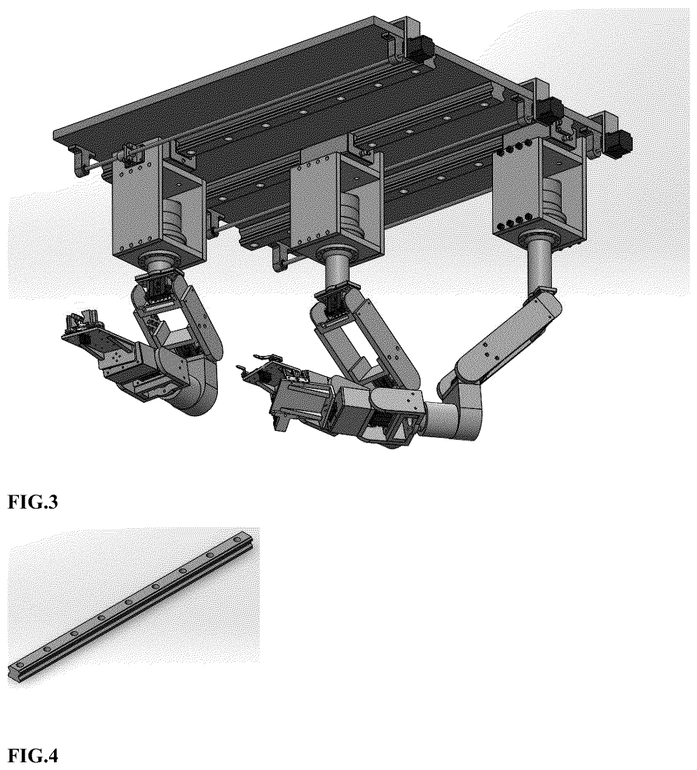

FIG. 3 is the bottom side of assembly presents a view of the underside of the robotic assembly. It highlights the lateral mobility of the three robotic arms, which allows them to move left or right as needed for different stages of the operation. This flexibility is achieved through the use of carriages and guideways that facilitate smooth and precise movement. Detailed mechanisms such as ball screws are used to convert the rotational motion provided by stepper motors into linear movement. These stepper motors, identifiable as the three black components on the right side of the assembly, ensure that each arm can be positioned with high precision. This coordinated movement is vital for performing intricate procedures without the arms interfering with each other's space.

FIG. 4 is the sensor holder zooms in on the arm that holds the ultrasonic sensor. This arm, like the others, features a 6-axis design, allowing for extensive maneuverability. The core components of this arm include a Harmonic drive, various metal and plastic structural elements, and servo motors which enable precise adjustments of the ultrasonic sensor's position. The Harmonic drive is significant as it reduces the gear ratio, thereby enhancing the accuracy of the arm's movements. This precision is essential for obtaining detailed and reliable skin assessments.

FIG. 5 is the harmonic drives focuses on the Harmonic drives used in the robotic system. These drives are crucial for attaining maximum precision by significantly reducing the gear ratio. The integration of Harmonic drives in the robotic arms ensures that movements are both accurate and stable, which is particularly important for performing delicate procedures such as injections where millimeter-scale precision is necessary.

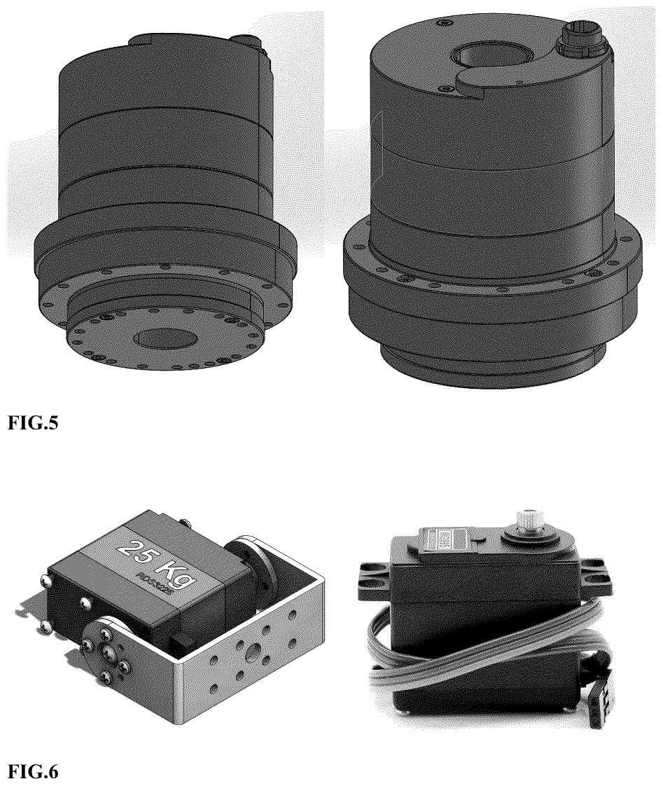

FIG. 6 is the servo motors elaborates on the servo motors employed within the robotic system. These motors are integral to the precise control of the robotic arms. Different types of servo motors are utilized to drive the various movements of the arms, ensuring that each can achieve the required positions and angles accurately. The reliability and precision of these servo motors are paramount for the system's overall performance.

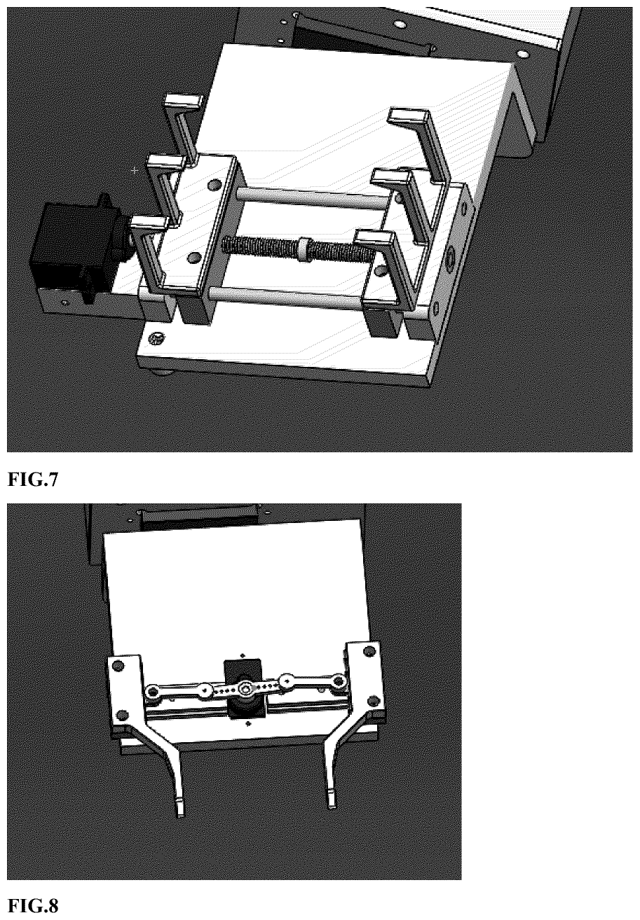

FIG. 7 is the ultrasonic sensor holder mechanism the seventh figure delves into the detailed mechanics of the ultrasonic sensor holder. This mechanism includes a reverse threaded rod which, when rotated by an MG 90 servo motor, allows the jaws holding the ultrasonic sensor to move closer together or farther apart. The internal threads of the jaws engage with the reverse threaded rod, enabling smooth and precise adjustments. This mechanism ensures that the ultrasonic sensor is held securely and can be rapidly repositioned as needed during the assessment process.

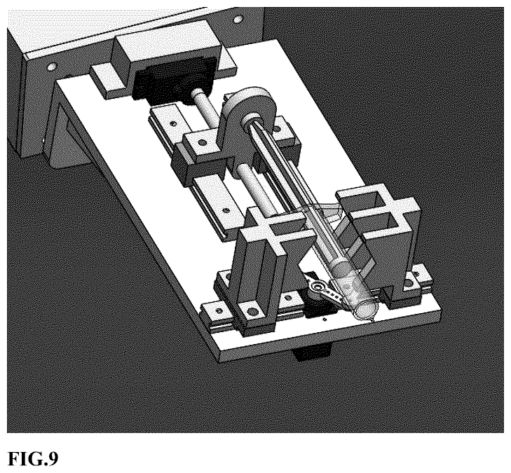

FIG. 8 is the pinching, holding, and pressing arm the eighth figure provides a detailed view of the middle arm, which is responsible for grabbing and stabilizing the skin. This arm also features a 6-axis design similar to the ultrasonic sensor holder. Its primary function is facilitated by a mechanism nearly identical to that of the sensor holder, but it is specifically tailored for pinching operations. This arm's jaws are designed to grip the skin securely, providing the necessary stabilization for precise injection.

FIG. 9 is the injection arm the ninth figure details the left arm, which holds the injection mechanism. This arm is designed to securely hold a syringe and administer the injectable substance. The holding and injecting process is controlled by a servo motor which pushes the plunger of the syringe in a controlled manner. This ensures that the correct dosage is administered at the precise depth and angle required for the procedure. The injection holder is built to accommodate standard syringes securely, ensuring they do not shift or dislodge during the procedure. Additionally, the servo motor control allows for fine-tuned movement of the plunger, enabling consistent and accurate delivery of the injectable substance. This design minimizes the risk of over or under-injection, thus enhancing patient safety and treatment efficacy.

Overall, each figure and its corresponding components collectively illustrate a sophisticated and highly precise surgical robotic system designed for non-surgical aesthetic treatments. The system's integration of advanced robotic technologies, such as Harmonic drives and servo motors, alongside specialized mechanisms for skin assessment and injection, ensures that the treatments are carried out with high precision, safety, and consistency. The mobility and versatility of the robotic arms, combined with the system's ability to be repositioned easily, make it an invaluable tool for practitioners aiming to deliver high-quality aesthetic care

The robotic system comprises three specialized arms, each serving a distinct and crucial function. Firstly, the injection arm is dedicated to administering aesthetic substances with exceptional precision. It is equipped with micromotor technology which enables ultra-precise needle movements for accurate placement and dosage. Additionally, the arm is integrated with sensors that measure and control the exact amount of substance injected, thereby significantly reducing the risk of over-injection or inaccurate placement. For example, in Botox treatments, this arm can be programmed to inject at specific facial points, ensuring consistent and optimal results.

The second component of the system is the skin grasping arm, which plays a vital role in stabilizing and manipulating the skin during procedures. This arm features an adaptive grasping mechanism that can adjust to various skin textures and thicknesses, ensuring firm but gentle handling of the skin. This functionality is especially beneficial during dermal filler procedures, where skin stability is essential for uniform injection and minimizing patient discomfort. By holding the skin firmly in place, the skin grasping arm enhances the accuracy of the injections, leading to more consistent aesthetic outcomes.

The third arm in the system is the ultrasound arm, designed to provide real-time imaging of the treatment area. This arm is equipped with wireless ultrasound technology that offers detailed, real-time internal imaging, allowing practitioners to identify and avoid danger zones. The arm's adjustable frequency settings enable customized imaging based on the specific area being treated. For instance, before injecting fillers, the ultrasound arm can map out facial artery locations, preventing accidental damage to critical blood vessels, thereby substantially enhancing the safety of the procedure.

One of the groundbreaking features of this robotic system is its capability for remote operation. This feature enables doctors to perform aesthetic procedures from a different location, thus overcoming geographical limitations. An on-site operator assists with basic tasks such as turning the robot on and off, positioning the needle and syringe, and disinfecting the injection arm. Integrated cameras and communication tools allow the doctor to monitor the procedure in real-time, communicate with the patient, and make necessary adjustments. This remote operation capability broadens the accessibility of high-quality aesthetic treatments to patients in remote or underserved areas, offering unprecedented flexibility for both practitioners and patients.

Safety is a paramount consideration in the design of this robotic system. The real-time imaging provided by the ultrasound arm significantly enhances safety by helping practitioners avoid critical structures during injections. An emergency stop mechanism is incorporated into the system, allowing for the immediate termination of the procedure if any anomaly or risk is detected. Additionally, the advanced sensors in the injection arm ensure precise dispensing of aesthetic substances, further minimizing the risk of complications such as vascular occlusion or uneven results.

To operate the RTA25 system, practitioners and operators must follow a series of steps designed to ensure safe, effective, and efficient treatment delivery. The detailed operation process includes:

The operator initializes the RTA25 system by switching it on and performing a preliminary check to ensure all components are functional. Syringes are placed or replaced in the rotating hand mechanism according to the specific treatment requirements. The system is disinfected thoroughly to maintain hygiene standards.

The client is positioned in view of the high-definition camera integrated with the RTA25 system. The practitioner uses the software to verify the client's treatment plan and review previous treatment records, if available.

The practitioner establishes video and audio communication with the client, ensuring clear and uninterrupted interaction. The consultation includes discussing the treatment plan, addressing client concerns, and confirming the procedural steps.

Using the remote-control tool, the practitioner performs the aesthetic treatment remotely. The tool's interface allows for precise control over injection depth, speed, and direction. The rotating hand mechanism, guided by the practitioner, administers the injections with high precision, emulating the movements of a skilled hand.

Throughout the procedure, the ultrasound module scans the treatment area. Any detected danger zones trigger immediate alerts, allowing the practitioner to adjust the treatment plan as needed. This continuous monitoring enhances the safety and effectiveness of the procedure.

The practitioner evaluates the treatment results in real-time through video interaction with the client. Before-and-after images are captured and stored in the central database for future reference and analysis. The practitioner provides post-procedure care instructions and schedules any necessary follow-up treatments. After the treatment, the operator performs routine maintenance tasks, including cleaning and disinfecting the rotating hand mechanism and remote-control tool. The operator ensures that all system components are in optimal working condition and ready for the next client.

By eliminating the limitations imposed by geographical barriers, the RTA25 facilitates wider access to high-quality aesthetic treatments, allowing practitioners to extend their services to underserved areas without the need for extensive travel. This not only enhances the convenience and efficiency for practitioners but also improves the accessibility of aesthetic care for clients worldwide. The combination of precise robotic technology, real-time control, and integrated safety features positions the RTA25 as a groundbreaking innovation that sets new standards in the field of aesthetic medicine, offering a reliable, efficient, and safe solution for modern aesthetic practices.

By addressing the critical needs of safety, accessibility, and efficiency, the RTA25 represents a transformative leap in the practice of aesthetic medicine. It harnesses advanced technology to offer a sophisticated solution that meets the evolving demands of both practitioners and clients, embodying the future of aesthetic treatments through its precision, innovation, and intelligent operation.

This invention offers several advantages over existing technologies. Unlike existing robotic systems that are primarily designed for invasive surgeries, this technology caters specifically to non-surgical aesthetic treatments. The three specialized robotic arms work in synchrony to deliver unparalleled precision, ensuring consistent and high-quality outcomes. The incorporation of real-time ultrasound imaging and precise control mechanisms dramatically reduces the risk of complications. By enabling remote procedures, the system overcomes geographical limitations, allowing patients from various locations to access top-tier aesthetic treatments.

In conclusion, this novel robotic system marks a significant advancement in the field of aesthetic medicine. By integrating specialized robotic arms with the capacity for remote operation and enhanced safety features, the system provides a unique solution to the existing challenges in non-surgical aesthetic procedures. This invention stands to revolutionize aesthetic medicine, offering improved precision, safety, and accessibility, and fulfilling a critical unmet need in the industry.

1. A remote-controlled aesthetic treatment system, comprising:
a rotating hand mechanism equipped with multiple motors and gears configured to perform precise and versatile movements for administering aesthetic treatments,
a remote-control tool designed to resemble a traditional syringe and configured to control the rotating hand mechanism's movements, including injection depth, speed, and direction,
an integrated ultrasound module within the rotating hand mechanism for real-time monitoring of treatment areas to detect critical structures and provide alerts to ensure safety during procedures,
a software platform that facilitates real-time video and audio communication between the practitioner and the client, manages a central database for storing client information and treatment records, and includes training modules for certifying practitioners and operators in the use of the system.

1.1. The system of claim 1, wherein the rotating hand mechanism is configured to accommodate and securely mount various types of needles and syringes, allowing for customization based on treatment requirements.

1.2. The system of claim 1, wherein the motors and gears of the rotating hand mechanism are controlled by software algorithms that provide smooth and accurate movement translations based on the inputs from the remote-control tool.

1.3. The system of claim 1, wherein the remote-control tool includes tactile feedback mechanisms that mimic the responsiveness of manual injections to enhance practitioner accuracy and control.

1.4. The system of claim 1, wherein the ultrasound module generates immediate alerts upon detection of danger zones, enabling practitioners to make real-time adjustments to the treatment process.

1.5. The system of claim 1, wherein the software platform's central database includes functionality for storing before-and-after treatment images and allows practitioners to track client progress over time.

1.6. The system of claim 1, wherein the training modules within the software platform provide step-by-step instructions and certification requirements for operating and maintaining the system.

2. A method for performing remote-controlled aesthetic treatments using the system claimed in claim 2, comprising:
initializing the system by performing preliminary checks and ensuring all components are functional,
securely mounting needles or syringes on the rotating hand mechanism and performing disinfection procedures,
positioning the client within the view of the system's high-definition camera,
establishing real-time video and audio communication between the practitioner and the client using the software platform,
performing the aesthetic treatment by remotely controlling the rotating hand mechanism with the remote-control tool, and making adjustments based on real-time ultrasound monitoring data,
capturing and storing before-and-after treatment images in the central database,
providing the client with post-procedure care instructions and scheduling follow-up treatments,
performing routine maintenance tasks on the system components to ensure readiness for subsequent treatments.

2.1. The method of claim 2, wherein the initialization includes verifying the secure wireless connection between the remote-control tool and the rotating hand mechanism.

2.2. The method of claim 2, wherein the step of securely mounting needles or syringes includes selecting the appropriate type based on the specific treatment requirements.

2.3. The method of claim 2, wherein the step of performing the aesthetic treatment includes receiving real-time alerts from the ultrasound module and making necessary adjustments to avoid critical structures.

2.4. The method of claim 2, wherein the software platform's video and audio communication feature facilitate thorough consultations and enables the practitioner to monitor the client's response during the treatment.

2.5. The method of claim 2, wherein the central database functionality includes the capability to generate analytic reports based on stored client records, assisting practitioners in optimizing treatment strategies.

2.6. The method of claim 2, wherein the routine maintenance tasks include cleaning and disinfecting the rotating hand mechanism and remote-control tool, and verifying the operational status of all system components.
